(19) United States
(12) Patent Application Publication
(10) Pub. No.: US 2008/0118579 A1
(43) Pub. Date: May 22, 2008

(54) Medical Device and Its use

(75) Inventors: Hannu Jarvelainen, Turku (FI); Matti Laato, Nousiainen (FI); Jukka Salonen, Turku (FI); Erik Vedel, Parainen (FI)

Correspondence Address: JAMES C. LYDON, 100 DAINGERFIELD ROAD, SUITE 100, ALEXANDRIA, VA, 22314, US

(73) Assignee: VIVOXID OY, Turku (FI)

(21) Appl. No.: 11/666,297

(22) PCT Filed: Nov. 2, 2005
(86) PCT No.: PCT/FI05/00468

§ 371 Date: May 25, 2007

(30) Foreign Application Priority Data
Nov. 2, 2004 (EP) 04396075.6

(51) Int. Cl. A61K 33/06; A61P 17/02; A61K 33/00; A61K 33/32; A61K 31/69; A61K 31/665

(52) U.S. Cl. 424/688; 424/724; 424/692; 424/722; 514/64; 514/100

(57) ABSTRACT

The invention relates to a medical device comprising non-sintered bioactive glass particles or fibres having a diameter in the range 5-100 μm, bioactive glass comprising SiO₂, Na₂O, CaO, K₂O, MgO, B₂O₃ and P₂O₅, wherein the amount of SiO₂ is 50-65 wt-% of the final total weight, Na₂O is 5-26 wt-% of the final total weight, CaO is 10-25 wt-% of the final total weight, K₂O is 0-15 wt-% of the final total weight, MgO is 0-6 wt-% of the final total weight, B₂O₃ is 0-4 wt-% of the final total weight, and P₂O₅ is 0-4 wt-% of the final total weight, provided that the total amount of Na₂O and K₂O is 10-30 wt-% of the final total weight The device is essentially drug free. The invention also relates to the use of said composition for treating lesions associated with compromised or poor vascularisation and for preventing avascular fibrosis.

### FIELD OF THE INVENTION

This invention relates to a medical device. The invention also relates to the use for treating lesions associated with compromised or poor vascularisation.

### BACKGROUND OF THE INVENTION

The publications and other materials used herein to illuminate the background of the invention, and in particular, the cases to provide additional details respecting the practice, are incorporated by reference.

The treatment of lesions, for example skin lesions, associated with compromised or poor vascularisation or blood perfusion is a demanding and difficult area of medicine. Improvement of angiogenesis, i.e. the new capillary blood vessel formation, is particularly important when natural healing is slow or is rendered difficult by a number of negative factors associated with e.g. infection of the wound, impeded blood flow, burns or other types of tissue damage/injury, medical treatment with cell poisons or steroids of various kind, or in cases when the patient suffers from chronic disorders with concomitant impairment of normal wound healing, i.e. when he or she is bedridden for prolonged periods of time, is of old age, has a cancer disease giving rise to serious nutritional deficiencies, has chronic inflammatory conditions of the intestine, has diabetes, is affected by conditions caused by atherosclerosis or venous diseases, and comparable types of conditions.

Currently the treatment of skin lesions mentioned above can be very problematic and places an enormous drain on the health resources. Recently various new approaches have been tested, including topical growth factors, e.g. platelet-derived growth factor, transforming growth factor β, granulocyte macrophage-colony stimulating factor, autologous skin grafts and bioengineered skin equivalents, e.g. Alloderm®, Integra®, Dermagraft-TC®, Apligraft®. The drawback of the skin replacements is their price, as they can be extremely expensive. Also, the polymer-based matrices can contain small amounts of monomers or polymerisation catalyst residues that can be detrimental or even toxic to the tissues.

Furthermore, interest is presently focused on angiogenesis induced e.g. by vascular endothelial growth factor delivered by gene therapy. However, at present these methods are not suitable for common use in clinical practice.

Another problem encountered in this field is the formation of an avascular fibrous capsule around an implanted biomaterial device and the mass transfer resistance associated with this fibrous capsule.

The use of bioactive glasses in medicine is now widely known. In this application, by bioactive glass it is meant a material that has been designed to induce specific biological activity in body tissues. Bioactive glasses react in aqueous systems and develop layers on their surfaces resulting in bonding between the device and the host tissue, but also release some of its component ions into the tissue. These ions can drift several hundred micrometers from the device surface. Unlike most other bioactive materials, the rate of chemical reactions of bioactive glasses can be controlled by changing the chemical composition of the glass.

US 2001/041186 discloses a composition comprising bioactive glass and a topical antibiotic for treating wounds and burns. The use of antibiotic can pose a problem in prolonged use, especially when the general trend in the medical field has been to reduce the use of antibiotics in order to minimise the risk of formation of antibiotic resistant bacterial strains. The preferred composition of the bioactive glass used in the said publication is so called “fast” bioactive glass, which means that it shows rapid surface reactions when coming into contact with body fluids. The fast surface reactions may cause cauterisation of the tissue that is treated with the composition.

WO2004/071542 discloses a bioactive material for use in stimulation of vascularisation. The bioactive material is used as formulation, ligature, tissue construct or as a coating on a dressing. The preferred glass composition of the publication is a ‘fast’ bioactive glass and it is used in amounts that vary between 0.00001 and 10 weight-%.

### OBJECTS AND SUMMARY OF THE INVENTION

The object of the invention is therefore to minimise or even eliminate the problems existing in the prior art.

Another object of this invention is to provide a medical device that can be used for treatment of lesions associated with compromised or poor vascularisation. Examples of such lesions are given above.

A typical medical device according to the present invention comprises non-sintered bioactive glass particles or fibres having a diameter in the range of 5-100 μm, the bioactive glass comprising SiO₂, Na₂O, CaO, K₂O, MgO, B₂O₃ and P₂O₅, wherein the amount of
SiO₂ is 50-65 wt-% of the final total weight,
Na₂O is 5-26 wt-% of the final total weight,
CaO is 10-25 wt-% of the final total weight,
K₂O is 0-15 wt-% of the final total weight,
MgO is 0-6 wt-% of the final total weight,
B₂O₃ is 0-4 wt-% of the final total weight, and
P₂O₅ is 0-4 wt-% of the final total weight,
   provided that the total amount of Na₂O and K₂O is 10-30 wt-% of the final total weight and that the device is essentially drug free.

The present invention also relates to the medical use of said bioactive glass composition for treating lesions associated with compromised or poor vascularisation.

### DETAILED DESCRIPTION OF THE INVENTION

The present invention thus relates to a medical device comprising a bioactive glass particles or fibres as disclosed above. The medical device is thus a vascularisation and healing promoting device. Other embodiments of the present invention are disclosed in the dependent claims.

Now it has been surprisingly found out that when the composition and the form of bioactive glass are selected according to the present invention in such a manner that the reaction rate on the surface of the glass is reduced, the capacity of the bioactive glass to promote vascularisation is improved. It is assumed, without wishing to be bound by a theory, that the more controlled rate of surface reactions inhibits harmful swaying of the local pH values in the lesion or wound.

The bioactive glasses used in the present invention also dissolve congruently in liquids, such as body fluids. Such bioactive glasses provide a burst of ions when in contact with the lesion, i.e., in contact with the tissue to be engineered. The medical device according to invention can thus prevent local depletion of the essential bioactive components in poorly vascularised tissue. These components may be provided by intravenous alimentation, but they can reach the injured tissue only if it is well vascularised. If the vascularisation in the injured tissue is lacking, a local depletion of the essential bioactive elements or components occurs, even if a solution containing these elements were given to the patient intravenously. The depletion of essential elements can be avoided when medical device according to the present invention is used in treatment of lesions or ulcers. The present invention therefore fulfils the long felt need for material, which is effective in promoting the revascularisation and at the same time easy to use and cost-effective. The bioactive glass according to the present invention thus stimulates cell growth and angiogenesis as described in this application.

The present invention provides a medical device that can be used for treatment of lesions associated with compromised or poor vascularisation as well as for induction of neovascularised fibrotic capsule. The device can thus be used for treatment of lesions caused by conditions due to lack of vascularisation or due to pathological changes of the vascularisation. It can also be used in association with drug delivery devices, cell encapsulation devices, tissue engineered neo-organ formation and implanted sensors that need proper vascularisation in order to function. The important point is not only the quantity of blood vessels, such as capillaries, arterioles and venules, but also their structural quality at the compromised site.

By compromised or poor vascularisation at the site of a lesion it is meant blood perfusion that has either deteriorated from the original, normal vascularisation, or that has not developed correctly, that is, up to the normal level, i.e., lacking vascularisation. By normal vascularisation it is meant the physiological vascularisation normally taking place in a healthy person.

The medical device according to the present invention is drug free. This means that the device does not comprise or include any synthetical molecules or compounds that are used in medication, such as antibiotics, anti-inflammatory agents, antiviral, anesthetic or analgesic agents. According to one embodiment the device does not comprise silver oxide AgO or other silver compounds that are sometimes used as antibacterial or antimicrobial agents.

The device according to the present invention thus comprises said bioactive glass composition. This means that the device may also comprise other materials. It may for example comprise additives. Such additives are for example selected from the group comprising biologically active agents, cellulose materials, cotton, other bioactive glasses, polymers or other structure supporting materials. The biologically active agents that are used in the present invention are compounds and agents that are occurring in the nature, for examples compounds and agents that exist as the products or intermediates of the metabolic pathway of a mammal, preferably human. The biologically active agents may have, of course, been produced synthetically, but they are essentially compounds and agents that exist as such in the nature. An example of such compound or agent is hexose sugar.

According to one preferred embodiment of the present invention the proportion of bioactive glass of the total weight of the medical device is typically more than 40 weight-%, more typically more than 50 weight-%, most typically more than 60 weight-%, preferably more than 70 weight-%, more preferably more than 80 weight-%, even more than 90 weight-%, sometimes even more than 95%, eventually about 100 weight-%. Typically the proportion of bioactive glass of the total weight of the medical device is typically less than 100 weight-%, sometimes less than 90 weight-%, occasionally less than 80 weight-%, at times even less than 70 weight-%. The high bioactive glass content of the medical device according to the present invention improves the control of the pH at the application site, e.g. in the diabetic ulcer. The high bioactive glass content makes it easier to maintain the optimal target pH for a longer period of time and with more accuracy.

The bioactive glass composition may be used in any suitable form, preferably though in the form of fibres or particles in the medical device according to the present invention. The composition may be used in the form of a woven or non woven fabric. When fibres are used, their diameter is typically in the range of 10-100 μm, more typically 15-50 μm, preferably 20-40 μm. On the other hand, when particles are used, the diameter of the particles is typically below 90 μm, even more typically below 45 μm. The diameter of the particles is typically and preferably over 5 μm, even more typically and preferably over 15 μm. The mean diameter of the particles is often 20-25 μm. When the medical device is used in treatment of full-grown humans it is advantageous that the main part of the particles have a diameter that is not smaller than 5 μm, as the particles smaller than this value may enter the capillaries and be diffused away from the application site. If the medical treatment is used for treatment of other types of mammals, the minimum diameter of the particles has to be determined by the capillary size of that mammal type.

According to one embodiment of the invention when bioactive glass particles are used in the medical device typically 10% of the glass particles have a diameter less than 4 μm, 20% of the glass particles have a diameter less than 7 μm, 30% of the glass particles have a diameter less than 12 μm, 40% of the glass particles have a diameter less than 17 μm, 50% of the glass particles have a diameter less than 23 μm, 60% of the glass particles have a diameter less than 29 μm, 70% of the glass particles have a diameter less than 35 μm, 80% of the glass particles have a diameter less than 40 μm, 90% of the glass particles have a diameter less than 48 μm, 95% of the glass particles have a diameter less than 55 μm.

According to another embodiment of the invention when bioactive glass particles are used in the medical device typically 10% of the glass particles have a diameter less than 5 μm, 20% of the glass particles have a diameter less than 10 μm, 30% of the glass particles have a diameter less than 15 μm, 40% of the glass particles have a diameter less than 20 μm, 50% of the glass particles have a diameter less than 25 μm, 60% of the glass particles have a diameter less than 30 μm, 70% of the glass particles have a diameter less than 35 μm, 80% of the glass particles have a diameter less than 40 μm, 90% of the glass particles have a diameter less than 50 μm, 95% of the glass particles have a diameter less than 55 μm.

When the bioactive glass is used in the particle form in the medical device, the particles may be bound together by a suitable binding agent, such as collagen, alginate, polysaccharides such as starch or hexose sugars, or polyvinylpyrrolidone. It is also naturally possible to use several different bioactive glass compositions in one device.

The device according to the present invention itself may have any size and shape desired. It may be in the form of granules, powder, paste, woven or non woven sheet, bandage, mat, adhesive plaster, pad or Band-Aid. It may be for example a large sheet used for treating pressure sores or a small sheet or in powder form for treating wounds associated with diabetes and/or other conditions exhibiting compromised or poor vascularisation. When the bioactive glass is used in powder form, it may be then covered with any known bandage.

According to one embodiment of the invention the medical device is constructed by embedding the particles of the bioactive glass in a supporting matrix, such as a cellulose material or a polymer. The cellulose material may for example be carboxymethyl cellulose, biomineralized cellulose or biomineralizable cellulose. The polymer may be any polymer suitable per se, such as polyolefins or polyesters. The particles of the bioactive glass can be homogenously embedded in the whole matrix, or they may be situated on the outer surface of the supporting matrix. According to one embodiment a mat woven from bioactive glass fibres is placed and attached on the supporting matrix made out of cellulose or polymer. The matrix can be in the form of a block, sheet or bandage.

Preferably the device according to the present invention is intended for daily use. This means that if the device is, for example, in paste form, the paste is applied daily to the application site, such as a diabetic ulcer or like.

According to one preferred embodiment the device is in form of solution, gel or paste. According to one especially preferred embodiment is the device is injectable, i.e. the device comprising bioactive glass is in form of a solution that can be injected into the wound, ulcer or the cavity to be treated. This enables that even the most remote parts of the irregular wounds, ulcers or cavities are reached and treated. In other words, the medical device according the invention can especially be used in treatment of infected tissue cavities, which are otherwise difficult to treat. The orally administered antibiotics do not reach these tissues via blood circulation if the tissues are poorly vascularised. The medical device supports the body's own mechanisms to overcome the infection in the tissue. At the same time, as the use of the device improves the revascularisation of the tissue, also orally administered antibiotics will reach it.

When the medical device is in solution form, the viscosity of the solution may be selected depending on the wound, ulcer or lesion to be treated. If the lesion is very deep and labyrinth-like cavity the medical device may be in form of thin and low-viscous solution in order to be sure that all the different parts of the lesion are reached. If the lesion is not so deep and more regular in form, a thicker and more viscous solution can be used. The medical device in solution, gel or paste form is easily prepared by adding to the bioactive glass powder a suitable volume of physiological salt solution or distilled water.

In the bioactive glass compositions of this application, the amount of different oxides is given as weight percent of the final total weight.

It is obvious to a person skilled in the art that the amounts of the oxides in the bioactive glass compositions can be freely chosen within the above-mentioned limits. Indeed, the amount of SiO₂ can be for example 51.5, 52, 53.5, 55, 56, 58, 61, 62.5 or 65 wt-% of the final total weight, the amount of Na₂O can be for example 5, 5.5, 6.2, 7, 7.3, 7.7, 8, 8.5, 9, 12, 15, 16.5, 18, 20.4, 24, 25.1 or 26 wt-% of the final total weight, the amount of CaO can be for example 10, 12.5, 14.7, 15, 16.5, 17, 20.3, 21, 21.4, 21.7, 22, 22.6, 23 or 25 wt-% of the final total weight, the amount of K₂O can be for example 0, 1.2, 2.5, 5, 5.5, 6.2, 7, 7.3, 8, 10, 10.5, 10.6, 11, 11.3, 11.7, 12, 14.2 or 15 wt-% of the final total weight, the amount of MgO can be for example 0, 0.5, 1, 1.3, 1.9, 2.4, 2.7, 3.5, 4, 4.5, 5.2 or 6 wt-% of the final total weight, the amount of B₂O₃ can be for example 0, 0.4, 0.6, 0.9, 1, 1.2, 2.7, 3.5 or 4 wt-% of the final total weight, and the amount of P₂O₅ can be for example 0, 0.5, 0.7, 1, 1.2, 2.5, 3.2 or 4 wt-% of the final total weight.

The bioactive glass composition used in the present invention can be made for example either by melting or by sol-gel process. The latter leads to a more porous glass than the melt-process.

According to an embodiment of the present invention, the bioactive glass has the following composition:
SiO₂ is 52-54 wt-% of the final total weight,
Na₂O is 5-7 wt-% of the final total weight,
CaO is 21-23 wt-% of the final total weight,
K₂O is 10-12 wt-% of the final total weight,
MgO is 4-6 wt-% of the final total weight,
B₂O₃ is 0-2 wt-% of the final total weight, and
P₂O₅ is 0-1 wt-% of the final total weight.

This composition is of particular interest if the bioactive glass in the medical device is in the fibre form. The fibres may be used, for example, for formation of an adhesive plaster.

According to another embodiment of the present invention, the bioactive glass has the following composition:
SiO₂ is 59-61 wt-% of the final total weight,
Na₂O is 24-26 wt-% of the final total weight,
CaO is 10-12 wt-% of the final total weight,
K₂O is 0-1 wt-% of the final total weight,
MgO is 0-1 wt-% of the final total weight,
B₂O₃ is 0-3 wt-% of the final total weight, and
P₂O₅ is 1-4 wt-% of the final total weight.

According to yet another embodiment of the present invention, the bioactive glass has the following composition:
SiO₂ is 52-54 wt-% of the final total weight,
Na₂O is 22-24 wt-% of the final total weight,
CaO is 19-21 wt-% of the final total weight,
K₂O is 0-1 wt-% of the final total weight,
MgO is 0-1 wt-% of the final total weight,
B₂O₃ is 0-1 wt-% of the final total weight, and
P₂O₅ is 0-1 wt-% of the final total weight.

According to an especially preferable embodiment, the bioactive glass has the composition of
SiO₂ is 53 wt-% of the final total weight,
Na₂O is 23 wt-% of the final total weight,
CaO is 20 wt-% of the final total weight and
P₂O₅ is 4 wt-% of the final total weight.

According to a still further embodiment of the present invention, the bioactive glass has the following composition:
SiO₂ is 52-61 wt-% of the final total weight,
Na₂O is 5-24 wt-% of the final total weight,
CaO is 10-23 wt-% of the final total weight,
K₂O is 0-12 wt-% of the final total weight,
MgO is 0-6 wt-% of the final total weight,
B₂O₃ is 0-3 wt-% of the final total weight, and
P₂O₅ is 0-4 wt-% of the final total weight,
   provided that the total amount of Na₂O and K₂O is 10-30 wt-% of the final total weight.

The present invention also relates to a bioactive glass composition comprising SiO₂, Na₂O, CaO, K₂O, MgO, B₂O₃ and P₂O₅, wherein the amount of
SiO₂ is 50-65 wt-% of the final total weight,
Na₂O is 5-26 wt-% of the final total weight,
CaO is 10-25 wt-% of the final total weight,
K₂O is 0-15 wt-% of the final total weight,
MgO is 0-6 wt-% of the final total weight,
B₂O₃ is 0-4 wt-% of the final total weight, and
P₂O₅ is 0-4 wt-% of the final total weight,
   provided that the total amount of Na₂O and K₂O is 10-30 wt-% of the final total weight and that the composition is essentially drug free, for treating lesions associated with compromised vascularisation.

The present bioactive glass composition can thus be used for treating lesions associated with compromised or poor vascularisation or promoting vascularisation of tissues associated with implanted biomaterial. The lesions include skin lesions or any other lesion or damaged tissue that exhibit delayed healing due to the lack of proper vascularisation or tendency for developing avascular fibrosis. In a typical treatment the lesion is firstly decontaminated with any known products, and then a device according to the present invention is applied on the lesion and preferably attached with gauze or the like. The device is left on place for the time needed for healing or changed at constant intervals, such as every day or once a week, depending on the characteristics of the lesion that is treated.

The invention further relates to a use of a bioactive glass composition comprising SiO₂, Na₂O, CaO, K₂O, MgO, B₂O₃ and P₂O₅, wherein the amount of
SiO₂ is 50-65 wt-% of the final total weight,
Na₂O is 5-26 wt-% of the final total weight,
CaO is 10-25 wt-% of the final total weight,
K₂O is 0-15 wt-% of the final total weight,
MgO is 0-6 wt-% of the final total weight,
B₂O₃ is 0-4 wt-% of the final total weight, and
P₂O₅ is 0-4 wt-% of the final total weight,
   provided that the total amount of Na₂O and K₂O is 10-30 wt-% of the final total weight, for the manufacture of an essentially drug free medical device for treating lesions associated with compromised or poor vascularisation.

The details given above in connection with the device apply also with respect to the uses.

In this specification, except where the context requires otherwise, the words “comprise”, “comprises” and “comprising” means “include”, “includes” and “including”, respectively. That is, when the invention is described or defined as comprising specified features, various embodiments of the same invention may also include additional features.

The invention is described below in greater detail by the following, non-limiting drawings.

### BRIEF DESCRIPTION OF THE DRAWINGS

FIG. 1 schematically illustrates a medical device according to a first embodiment of the present invention.

FIG. 2 illustrates the results of Example 5.

### DETAILED DESCRIPTION OF THE DRAWINGS

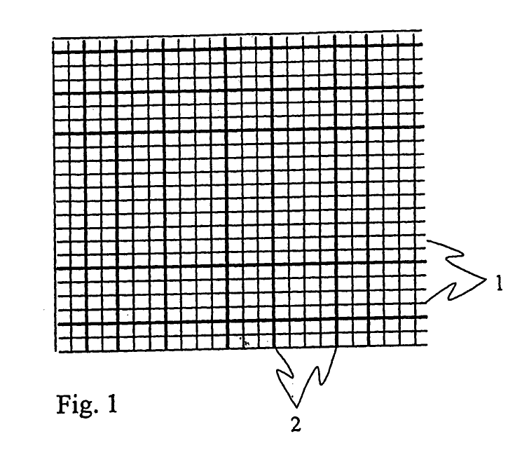

FIG. 1 schematically illustrates a medical device according to a first embodiment of the present invention. In this embodiment, the medical device is constituted of a woven tissue consisting essentially of fibers in two directions. The fibers 1 are made of a different bioactive glass composition that the fibers 2.

### EXPERIMENTAL PART

### Example 1

A composition consisting of:

158.25 g of SiO₂,

14.55 g of CaH(PO₄)×2H₂O,

98.63 g of CaCO₃,

102.60 g of Na₂CO₃,

16.51 g of K₂CO₃, and

4.50 g of MgO.

was heated to a temperature of 1360° C. and maintained at this temperature for a period of three hours. The melted composition wherein the carbonates had reacted forming oxides was allowed to cool down to ambient temperature overnight and the solid glass was crushed.

The crushed glass material was reheated to a temperature of 1360° C. and maintained in this temperature for a period of three hours. The resulting glass was cast into a mold and allowed to cool down to ambient temperature. 300 g of bioactive glass according to the present invention was obtained. The composition of the glass was the following:
SiO₂ 52.75 wt-%,
P₂O₅ 2.00 wt-%,
CaO 20.00 wt-%,
Na₂O 20.00 wt-%,
K₂O 3.75 wt-%, and
MgO 1.50 wt-%.

### Example 2

A composition consisting of:

159.00 g of SiO₂,

29.10 g of CaH(PO₄)×2H₂O,

90.16 g of CaCO₃ and

118.0 g of Na₂CO₃

was heated to a temperature of 1360° C. and maintained at this temperature for a period of three hours. The melted composition wherein the carbonates had reacted forming oxides was allowed to cool down to ambient temperature overnight and the solid glass was crushed.

The crushed glass material was reheated to a temperature of 1360° C. and maintained in this temperature for a period of three hours. The resulting glass was cast into a mold and allowed to cool down to ambient temperature. 300 g of bioactive glass according to the present invention was obtained. The composition of the glass was the following:
SiO₂ 53 wt-%,
P₂O₅ 4 wt-%,
CaO 20 wt-% and
Na₂O 23 wt-%,

### Example 3

A composition consisting of:

168.00 g of SiO₂,

14.55 g of CaH(PO₄)×2H₂O,

71.85 g of CaCO₃,

128.26 g of Na₂CO₃, and

10.66 g of H₃BO₃

was heated to a temperature of 1360° C. and maintained at this temperature for a period of three hours. The melted composition wherein the carbonates had reacted forming oxides was allowed to cool down to ambient temperature overnight and the solid glass was crushed.

The crushed glass material was reheated to a temperature of 1360° C. and maintained in this temperature for a period of three hours. The resulting glass was cast into a mold and allowed to cool down to ambient temperature. 300 g of bioactive glass according to the present invention was obtained. The composition of the glass was the following:
SiO₂ 56 wt-%,
P₂O₅ 2 wt-%,
CaO 15 wt-%,
Na₂O 25 wt-%, and
B₂O₃ 2 wt-%.

### Example 4

179.10 g of SiO₂,

18.19 g of CaH(PO₄)×2H₂O,

48.32 g of CaCO₃,

130.82 g of Na₂CO₃, and

6.93 g of H₃BO₃

was heated to a temperature of 1360° C. and maintained at this temperature for a period of three hours. The melted composition wherein the carbonates had reacted forming oxides was allowed to cool down to ambient temperature overnight and the solid glass was crushed.

The crushed glass material was reheated to a temperature of 1360° C. and maintained in this temperature for a period of three hours. The resulting glass was cast into a mold and allowed to cool down to ambient temperature. 300 g of bioactive glass according to the present invention was obtained. The composition of the glass was the following:
SiO₂ 59.7 wt-%,
P₂O₅ 2.5 wt-%,
CaO 11 wt-%,
Na₂O 25.5 wt-% and
B₂O₃ 1.3 wt-%.

Any of the above-mentioned bioactive glasses can be used for drawing of a fiber by standard methods.

### Example 5

The effect of the bioactive glass composition A (disclosed in Example 2) on stimulating angiogenesis was studied by using a standardized experimental wound model, i.e. in subcutaneously implanted viscose cellulose sponges in mice.

Viscose cellulose sponges (manufactured by Cellomeda Oy, Turku, Finland) was used as a framework matrix to study tissue repair. The material was cut into rectangular pieces, 20 mm long and 5 mm in diameter. The sponges were decontaminated by boiling for 30 minutes in physiological saline and the implantations were performed with strictly aseptic techniques. C57BL mice were anesthetized and a 2 cm long incision was made in the dorsal midline at the caudal portion of the back. Each mouse received two sponges that were implanted longitudinally under the skin, cephaled from the incision. During the experiments, the animals received a normal mouse diet and water ad libitum and were housed individually in cages in the animal quarters.

Viscose cellulose sponges were filled with 100 μl of a suspension containing 700 μl 0.9% NaCl solution, 0.5 g of the glass of Example 2 and 1 g of hexose sugars, or with 0.9% NaCl solution alone. Seventeen and 24 days after sponge implantations, the mice were sacrificed, and the sponges were harvested and analyzed for vascularity by counting the number of all erythrocyte-containing blood vessels (capillaries) in formalin-fixed, paraffin-embedded and haematoxylin and eosin stained histological sections of sponge specimens using light microscope (magnification 40×) as described in the publication Puolakkainen et al., 2003 (Puolakkainen P, Bradshaw A D, Kyriakides T R, Reed M, Brekken R, Wight T, Bornstein P, Ratner B, Sage E H. Compromised production of extracellular matrix in mice lacking secreted protein, acidic and rich in cysteine (SPARC) leads to a reduced foreign body reaction to implanted biomaterials. Am. J. Pathol. 2003; 162: 627-635).

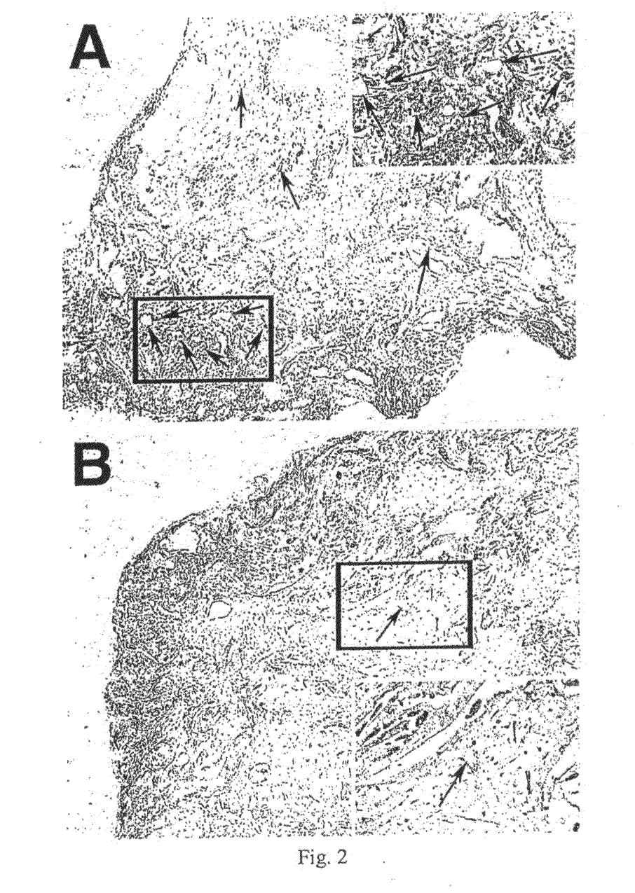

The results demonstrated that the sponges filled with bioactive glass and hexose sugars containing suspension exhibited markedly more blood vessels compared to the sponges filled with the suspension alone (see FIG. 2, in which representative pictures on sponges filled with bioactive glass containing suspension (panel A) or with the suspension alone (panel B) at the 24 day time point are shown. The arrows in the pictures indicate erythrocyte containing blood vessels). The above results indicate that bioactive glass has a stimulatory effect on new capillary blood vessel formation (i.e. angiogenesis), and thereby, bioactive glass is believed to be of great value when new strategies e.g. for impaired wound healing are designed.

1-8. (canceled)

9. A method for treatment of a lesion of a patient, comprising
administering a drug-free bioactive glass composition to a patient suffering from a lesion associated with compromised or poor vascularization,
wherein said bioactive glass composition is in the form of particles or fibers,
wherein said bioactive glass composition comprises SiO₂, Na₂O, CaO, K₂O, MgO, B₂O₃ and P₂O₅ in the following weight percentage ranges based on the total weight of the composition:
50-65 weight percent of SiO₂,
5-26 weight percent of Na₂O,
10-25 weight percent of CaO,
0-15 weight percent of K₂O,
0-6 weight percent of MgO,
0-4 weight percent of B₂O₃, and
0-4 weight percent of P₂O₅,
provided that the total amount of Na₂O and K₂O is 10-30 weight percent of the total weight of the composition.

10. The method of claim 9, wherein the lesion is a skin lesion.

11. The method of claim 9, wherein the bioactive glass particles or fibers are non-sintered and have a diameter of from 5 to 100 um.

12. The method of claim 9, wherein said bioactive glass composition comprises SiO₂, Na₂O, CaO, K₂O, MgO, B₂O₃ and P₂O₅ in the following weight percentage ranges based on the total weight of the composition:
52-54 weight percent of SiO₂,
5-7 weight percent of Na₂O,
21-23 weight percent of CaO,
10-12 weight percent of K₂O,
4-6 weight percent of MgO,
0-2 weight percent of B₂O₃, and
0-1 weight percent of P₂O₅.

13. The method of claim 9, wherein said bioactive glass composition comprises SiO₂, Na₂O, CaO, K₂O, MgO, B₂O₃ and P₂O₅ in the following weight percentage ranges based on the total weight of the composition:
59-61 weight percent of SiO₂,
24-26 weight percent of Na₂O,
10-12 weight percent of CaO,
0-1 weight percent of K₂O,
0-1 weight percent of MgO,
0-3 weight percent of B₂O₃, and
1-4 weight percent of P₂O₅.

14. The method of claim 9, wherein said bioactive glass composition comprises SiO₂, Na₂O, CaO, K₂O, MgO, B₂O₃ and P₂O₅ in the following weight percentage ranges based on the total weight of the composition:
52-54 weight percent of SiO₂,
22-24 weight percent of Na₂O,
19-21 weight percent of CaO,
0-1 weight percent of K₂O,
0-1 weight percent of MgO,
0-1 weight percent of B₂O₃, and
1-1 weight percent of P₂O₅.

15. A method for manufacturing a drug-free medical device, comprising,
embedding particles or fibers of a bioactive glass composition in or to a supporting matrix,
wherein said bioactive glass composition comprises SiO₂, Na₂O, CaO, K₂O, MgO, B₂O₃ and P₂O₅ in the following weight percentage ranges based on the total weight of the composition:
50-65 weight percent of SiO₂,
5-26 weight percent of Na₂O,
10-25 weight percent of CaO,
0-15 weight percent of K₂O,
0-6 weight percent of MgO,
0-4 weight percent of B₂O₃, and
0-4 weight percent of P₂O₅,
provided that the total amount of Na₂O and K₂O is 10-30 weight percent of the total weight of the composition.

16. The method of claim 15, wherein the amount of bioactive glass composition in the medical device is more than 40 weight percent of the total weight of the device.

17. The method according to claim 15, wherein said device further comprises additives selected from the group consisting of biologically active agents, cellulose materials, cotton, other bioactive glasses and polymers.
